(19) United States
(12) Patent Application Publication
(10) Pub. No.: US 2018/0174687 A1
(43) Pub. Date: Jun. 21, 2018

(54) METHODS AND SYSTEMS FOR IMPROVING PERFORMANCE OF A HEALTHCARE PROVIDER

(71) Applicant: ABBOTT LABORATORIES, Abbott Park, IL (US)

(72) Inventors: Vahe Ayvazian, Vernon Hills, IL (US); Jonathan V. Burgart, Antioch, IL (US)

(73) Assignee: ABBOTT LABORATORIES, Abbott Park, IL (US)

(21) Appl. No.: 15/849,259

(22) Filed: Dec. 20, 2017

Related U.S. Application Data
(60) Provisional application No. 62/436,928, filed on Dec. 20, 2016.

(51) Int. Cl. G16H 50/70; G16H 50/30; G16H 20/40; G16H 70/20

(52) U.S. Cl. CPC G16H 50/70; G16H 70/20; G16H 20/40; G16H 50/30

(57) ABSTRACT

Determining recommended medical procedures for a medical indication of interest includes correlating a hospital performance ranking database with a patient medical history information database to determine, for each medical indication, a difference in a frequency of the at least one medical procedure performed by at least one hospital of the plurality of hospitals above a high ranking threshold compared to a frequency of the at least one medical procedure performed by at least one hospital of the plurality of hospitals below a low ranking threshold, selecting a medical indication of interest from the plurality of medical indications, and presenting to a user the at least one medical procedure having the difference in the frequency above the predetermined threshold for the medical indication of interest.

### CROSS-REFERENCE TO RELATED APPLICATIONS

This application claims the benefit of U.S. Provisional Application No. 62/436,928, filed Dec. 20, 2016, which is incorporated herein by reference in its entirety.

### BACKGROUND

The disclosed subject matter relates to systems and methods for improving performance of a healthcare provider, such as a hospital, and more particularly, to providing systems and methods for determining recommended medical procedures for a medical indication of interest.

Performance of a healthcare provider can be ranked based on a variety of metrics related to performance of a healthcare provider, including but not limited to, outcome metrics, such as patient readmissions, complications and/or mortality. Healthcare providers, such as hospitals, whose metrics, such as readmission rates, are below a satisfactory level can face penalties in the form of increased costs or fines. As such, improving the performance of a healthcare provider is desired.

Healthcare providers can face uncertainty in ordering and interpreting clinical laboratory tests. Indeed, diagnostic errors are a significant cause of malpractice claims and patient deaths. Yet, medical claim data can provide information about diagnostic testing and/or treatments used to diagnose and/or treat certain medical indications. Such medical claim data, however, does not provide a link between diagnostic testing and/or treatments to improved patient outcomes.

As such, there is an opportunity for electronic clinical decision support tools that can determine recommended medical procedures, including but not limited to diagnostic tests and/or treatments, for a medical indication of interest, that can improve performance of healthcare providers, including but not limited to, improved patient outcomes.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the drawings.

To achieve these and other advantages and in accordance with the purpose of the disclosed subject matter, as embodied and broadly described, the disclosed subject matter includes systems and methods for improving performance of a healthcare provider, such as a hospital. According to one aspect, the disclosed subject matter includes systems and methods for determining recommended medical procedures for a medical indication of interest.

In accordance with the disclosed subject matter, for purpose of illustration and not limitation, a method for determining recommended medical procedures for a medical indication of interest is provided. The method includes obtaining a hospital performance ranking database including a plurality of hospitals and, for each hospital, a ranking of the hospital relative to each other hospital in at least one metric related to hospital performance.

The method further includes obtaining a patient medical history information database including patient medical history information for a plurality of patients. The patient medical history information includes, for each patient, at least one medical procedure performed by a hospital of the plurality of hospitals in response to at least one of a plurality of medical indications. The method further includes correlating the hospital performance ranking database with the patient medical history information database. For example, and without limitation, the method can include correlating the hospital performance ranking database with the patient medical history information database to determine, for each medical indication, a difference in a frequency of the at least one medical procedure performed by at least one hospital of the plurality of hospitals above a high ranking threshold compared to a frequency of the at least one medical procedure performed by at least one hospital of the plurality of hospitals below a low ranking threshold. The method further includes identifying, for each medical indication, the at least one medical procedure having the difference in the frequency above or below a predetermined threshold.

Additionally or alternatively, as embodied herein, the method further includes obtaining a hospital interview response database including a plurality of medical procedures determined to reduce readmissions for at least one of a plurality of medical indications and, for at least one hospital of the plurality of hospitals, a response to whether the at least one hospital performs each of the plurality of medical procedures determined to reduce readmissions for the least one medical indication. The method includes identifying, from the hospital interview response database, at least one medical procedure of the plurality of medical procedures determined to reduce readmissions for the least one medical indication that is not performed by the at least one hospital.

The method further includes selecting, for the at least one hospital, a medical indication of interest from the plurality of medical indications. The method further includes presenting to a user, for the at least one hospital, at least one recommended medical procedure to improve the at least one metric related to hospital performance, the at least one recommended medical procedure selected from at least one of the at least one medical procedure having the difference in the frequency above the predetermined threshold for the medical indication of interest and the at least one medical procedure of the plurality of medical procedures determined to reduce readmissions for the least one medical indication not performed by the at least one hospital.

Additionally or alternatively, the hospital performance ranking database can include, for each hospital, a history of rankings of the hospital relative to each other hospital in the at least one metric related to hospital performance and/or a sensitivity analysis of metrics for which the hospital is below average.

Additionally or alternatively, the user can be presented, for the at least one hospital, based on the history of rankings of the hospital in the at least one metric related to hospital performance, an estimated improvement in the at least one metric related to hospital performance if the at least one recommended medical procedure is implemented, and/or, based on the sensitivity analysis, an estimated cost savings if the at least one recommended medical procedure is implemented.

Additionally, and as embodied herein, the hospital performance ranking database can be formatted from a plurality of databases to comprise a single row of information for each hospital of the plurality of hospitals. The at least one metric can include at least one outcome metric, patient satisfaction metric, clinical care metric, patient safety metric, and hospital operations metric. The at least one outcome metric can include at least one of morality, readmissions, and complications. The ranking of each hospital can correspond to one of four quartiles.

Furthermore, the method can include providing a database of best practice information, including, for each of the hospitals of the plurality of hospitals, whether the hospital performs one or more best practices. The hospital can be further ranked based at least in part on whether the hospital performs one or more best practices. Additionally or alternatively, the method can further include identifying areas of need based on sensitivity analysis of metrics for which the hospital is performing below average. Furthermore, or as a further alternative, the method can include identifying areas of need based on questions based on best practices (e.g., based on a “Readiness Assessment”). In addition, or as an additional alternative, the method can include presenting, to a user, recommendations for improving identified areas of need based on the database of best practices (e.g., implementing RED as discharge protocol).

As embodied herein, and as set forth in more detail below, methods and systems for determining recommended medical procedures for a medical indication of interest are provided in accordance with the disclosed subject matter herein. An exemplary embodiment of a method for determining recommended medical procedures for a medical indication of interest is depicted in the drawings and described in further detail below.

It is to be understood that both the foregoing general description and the following detailed description are exemplary and are intended to provide further explanation of the disclosed subject matter.

The accompanying drawings, which are incorporated in and constitute part of this specification, are included to illustrate and provide a further understanding of the methods and systems of the disclosed subject matter. Together with the description, the drawings explain the principles of the disclosed subject matter.

### BRIEF DESCRIPTION OF THE DRAWINGS

The details of the subject matter set forth herein, both as to its structure and operation, may be apparent by study of the accompanying figures, in which like reference numerals refer to like parts.

FIGS. 1A-1B together are a flow diagram illustrating exemplary methods for determining recommended medical procedures for a medical indication of interest in accordance with embodiments of the disclosed subject matter.

FIG. 2 is a diagram illustrating additional details of exemplary methods for determining recommended medical procedures for a medical indication of interest in accordance with embodiments of the disclosed subject matter.

FIG. 3 is a diagram illustrating additional details of obtaining a hospital performance ranking database, obtaining a patient medical history information database, and correlating the hospital performance ranking database with the patient medical history information database for use with exemplary methods for determining recommended medical procedures for a medical indication of interest in accordance with embodiments of the disclosed subject matter.

FIG. 4 is a diagram illustrating additional details of obtaining a hospital performance ranking database for use with exemplary methods for determining recommended medical procedures for a medical indication of interest in accordance with embodiments of the disclosed subject matter.

FIG. 5 is a diagram illustrating additional details of obtaining a hospital interview response database for use with exemplary methods for determining recommended medical procedures for a medical indication of interest in accordance with embodiments of the disclosed subject matter.

FIG. 6 is a diagram illustrating additional details of obtaining a hospital performance ranking database for use with exemplary methods for determining recommended medical procedures for a medical indication of interest in accordance with embodiments of the disclosed subject matter.

FIG. 7 is a diagram illustrating additional details of obtaining a hospital performance ranking database for use with exemplary methods for determining recommended medical procedures for a medical indication of interest in accordance with embodiments of the disclosed subject matter.

FIG. 8 is a diagram illustrating additional details of obtaining a hospital performance ranking database for use with exemplary methods for determining recommended medical procedures for a medical indication of interest in accordance with embodiments of the disclosed subject matter.

FIG. 9 is a diagram illustrating additional details of obtaining a hospital performance ranking database for use with exemplary methods for determining recommended medical procedures for a medical indication of interest in accordance with embodiments of the disclosed subject matter.

FIG. 10 is a diagram illustrating additional details of presenting at least one recommended medical procedure to a user for use with exemplary methods for determining recommended medical procedures for a medical indication of interest in accordance with embodiments of the disclosed subject matter.

FIG. 11 is a diagram illustrating additional details of identifying at least one medical procedure determined to reduce readmissions for use with exemplary methods for determining recommended medical procedures for a medical indication of interest in accordance with embodiments of the disclosed subject matter.

FIG. 12 is a diagram illustrating additional details of obtaining a hospital interview response database for use with exemplary methods for determining recommended medical procedures for a medical indication of interest in accordance with embodiments of the disclosed subject matter.

### DETAILED DESCRIPTION

Reference will now be made in detail to the various exemplary embodiments of the disclosed subject matter, exemplary embodiments of which are illustrated in the accompanying drawings. The structure and corresponding method of operation of the disclosed subject matter will be described in conjunction with the detailed description of the system.

The systems and methods presented herein can be used for improving the performance of a healthcare provider. For purpose of illustration of the disclosed subject matter, and not limitation, reference is made herein to improving performance of a hospital. It is understood, however, that the systems and methods described herein can be used to improve performance of any healthcare provider, for example and without limitation, primary care providers, inpatient or outpatient clinics, medical laboratories, pharmacies, physical therapy clinics, or any other healthcare provider.

The disclosed subject matter thus relates to techniques for systems and methods for improving performance of a healthcare provider, such as a hospital, including, according to some aspects, systems and methods for determining recommended medical procedures for a medical indication of interest. The purpose and advantages of the disclosed subject matter will be set forth and apparent from the description that follows. Additional advantages of the disclosed subject matter will be realized and attained by the methods, apparatus, and devices particularly pointed out in the written description and claims thereof, as well as from the appended drawings.

In accordance with the disclosed subject matter, for purpose of illustration and not limitation, a method for determining recommended medical procedures for a medical indication of interest is provided. The method includes obtaining a hospital performance ranking database including a plurality of hospitals and, for each hospital, a ranking of the hospital relative to each other hospital in at least one metric related to hospital performance. The method further includes obtaining a patient medical history information database including patient medical history information for a plurality of patients. The patient medical history information includes, for each patient, at least one medical procedure performed by a hospital of the plurality of hospitals in response to at least one of a plurality of medical indications. The method further includes correlating the hospital performance ranking database with the patient medical history information database. For example, and without limitation, the method can include correlating the hospital performance ranking database with the patient medical history information database to determine, for each medical indication, a difference in a frequency of the at least one medical procedure performed by at least one hospital of the plurality of hospitals above a high ranking threshold compared to a frequency of the at least one medical procedure performed by at least one hospital of the plurality of hospitals below a low ranking threshold. The method further includes identifying, for each medical indication, the at least one medical procedure having the difference in the frequency above or below a predetermined threshold.

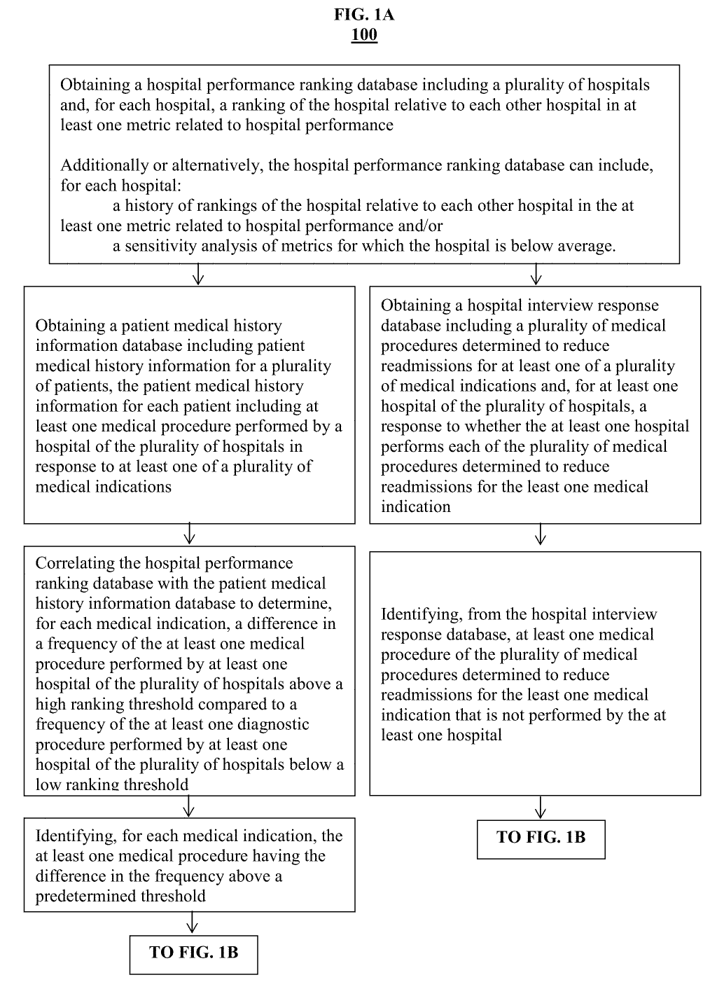

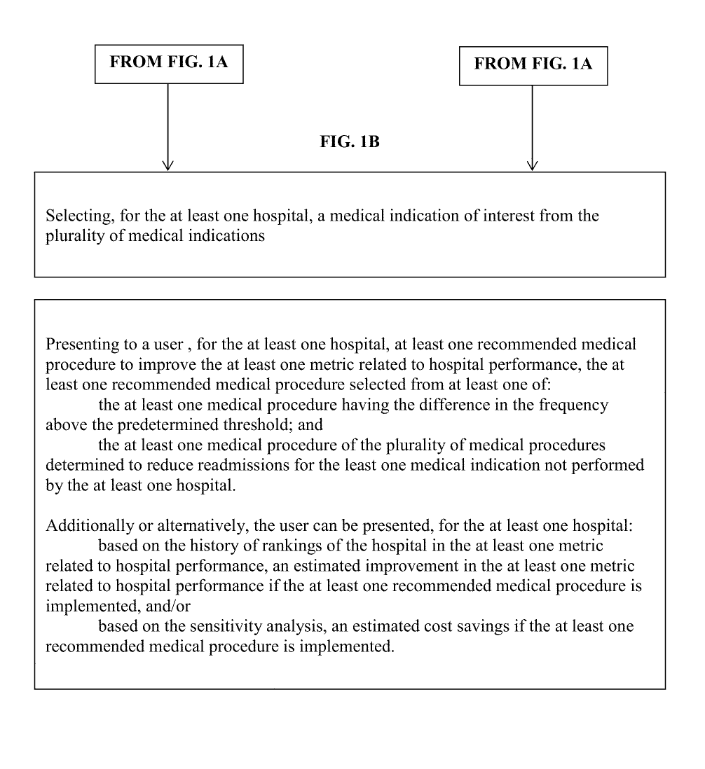

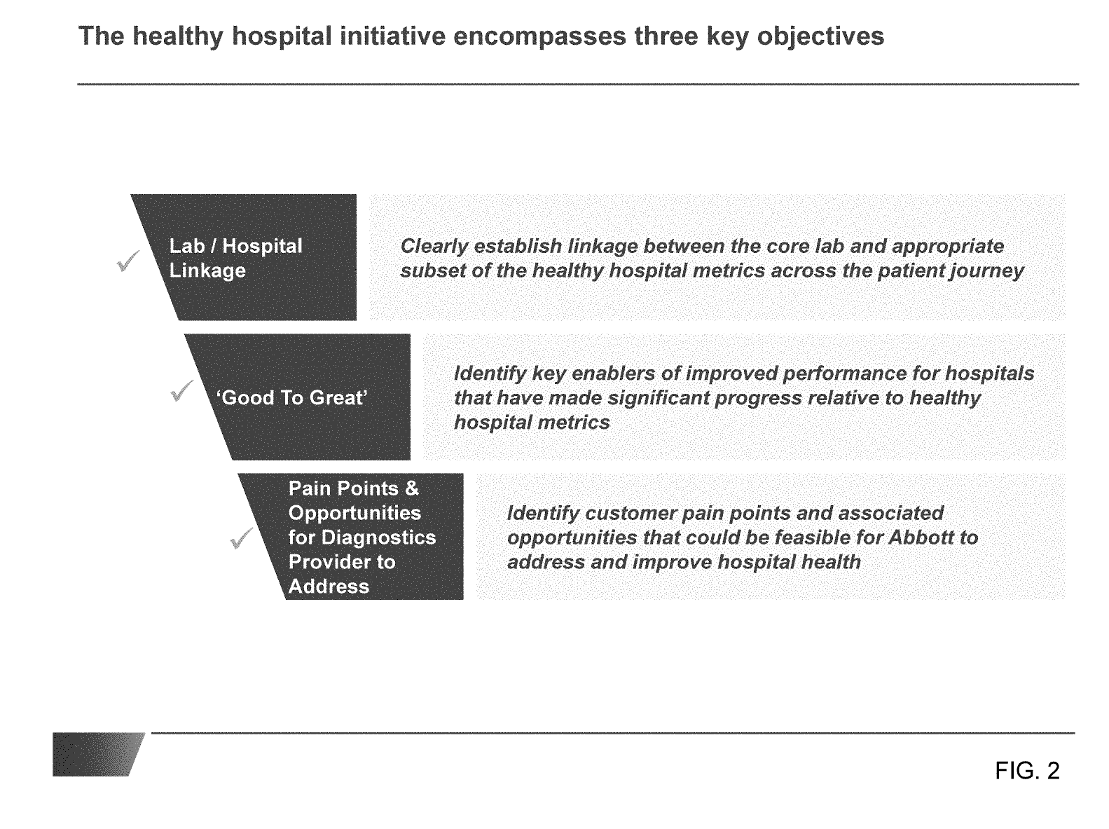

For purpose of illustration and not limitation, reference is made to the exemplary embodiment of a method 100 for determining recommended medical procedures for a medical indication of interest shown in FIGS. 1A-1B. With reference to FIG. 2, for purpose of illustration and not limitation, method 100 can be used to establish linkage between laboratory services and hospital performance metrics. Additionally or alternatively, method 100 can be used to identify procedures and/or practices leading to improved performance for hospitals that have made significant progress relative to hospital performance metrics. In addition or as a further alternative, method 100 can be used to identify poor hospital performance and associated opportunities to address and improve hospital performance.

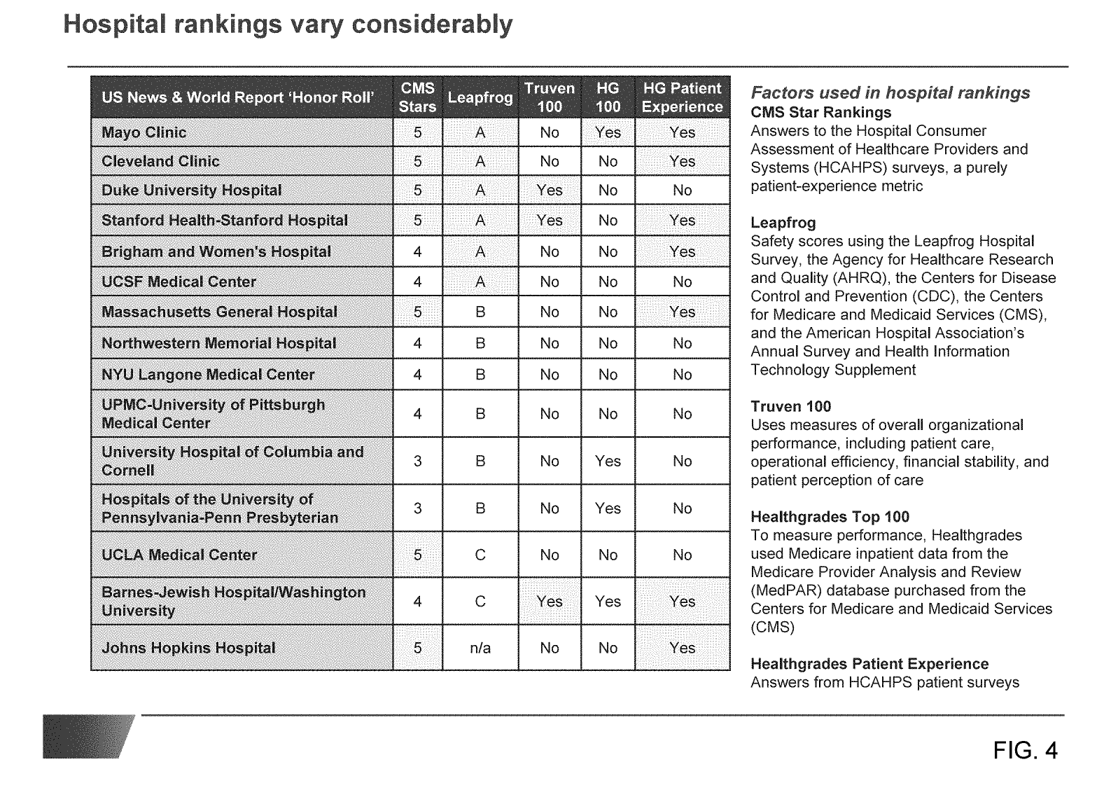

Referring again to FIG. 1A, for purpose of illustration and not limitation, method 100 includes, at 101, obtaining a hospital performance ranking database. The hospital performance ranking database can include, for example and without limitation, and as embodied herein, a plurality of hospitals and, for each hospital, a ranking of the hospital relative to each other hospital in at least one metric related to hospital performance. Additionally or alternatively, the hospital performance ranking database can include, for each hospital, a history of rankings of the hospital relative to each other hospital in the at least one metric related to hospital performance and/or a sensitivity analysis of metrics for which the hospital is below average. For purpose of illustration, and not limitation, FIG. 4 shows exemplary sources of hospital performance ranking databases for use with the systems and techniques described herein. For purpose of illustration and not limitation, as embodied herein, the hospital performance ranking database can include, or be obtained from, a publicly-available database. For example and without limitation, as embodied herein, the hospital performance ranking database can include data obtained from the Centers for Medicare & Medicaid Services (“CMS”) Hospital Quality Star Ratings data and/or the Veterans Health Administration Strategic Analytics for Improvement and Learning (“SAIL”).

Additionally, and as embodied herein, the hospital performance ranking database can be obtained from a plurality of databases, such as the publicly-available databases or portions of such databases identified herein, to select the desired metric data and format the selected data into a single row of information for each hospital of the plurality of hospitals. Such data can be obtained and formatted automatically, for example and as embodied herein, using formulas, scripts or macros, which can be customized based on the original format of the data to be obtained.

Furthermore, and as embodied herein, metrics related to hospital performance can include, for purpose of illustration and not limitation, at least one of an outcome metric, a patient satisfaction metric, a clinical care metric, a patient safety metric, and/or a hospital operations metric. For example, and as embodied herein, outcome metrics can include, for purpose of illustration and not limitation, at least one of mortality, readmissions and complications. As embodied herein, the ranking of each hospital can correspond to one of four quartiles, ranging from top performing hospitals to worst performing hospitals.

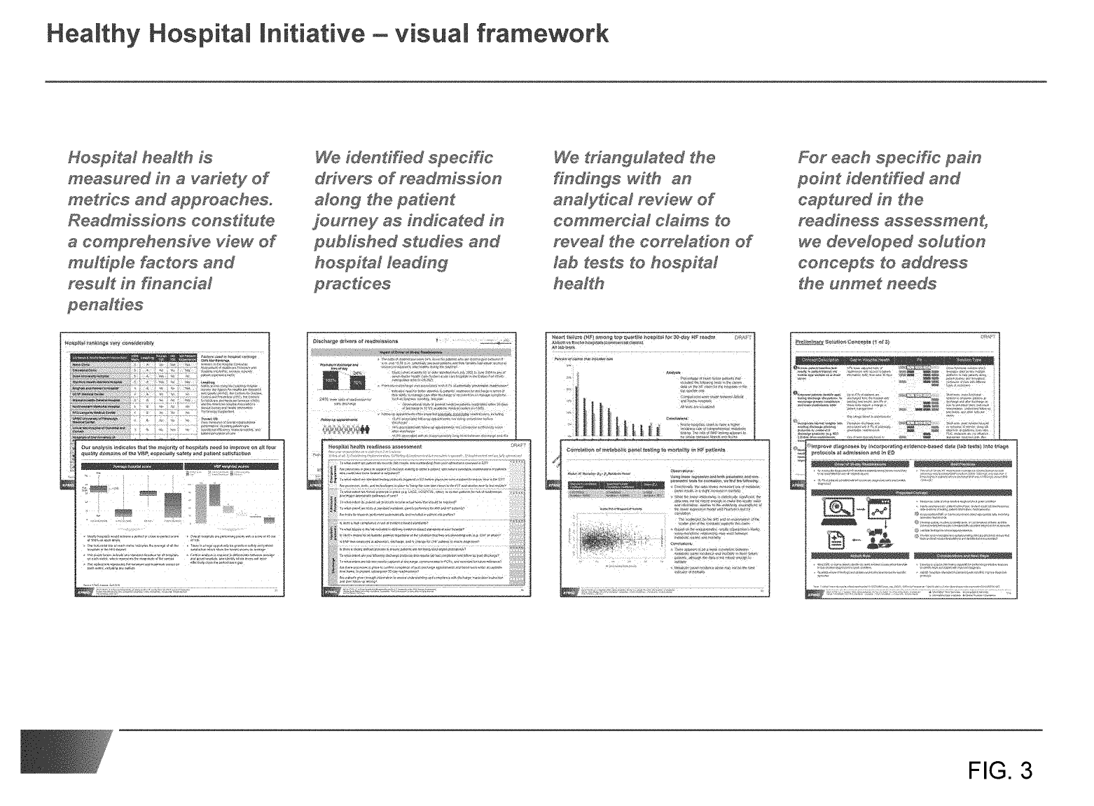

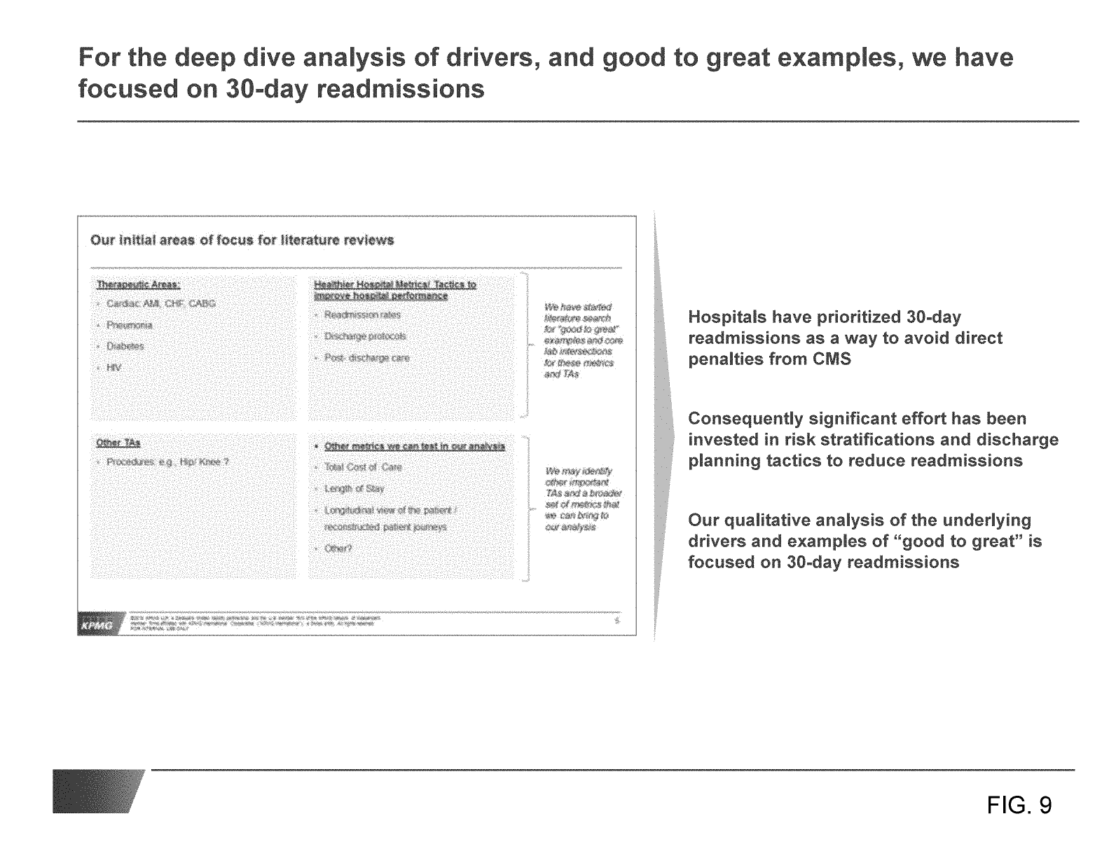

Certain metrics can impact the financial performance of hospitals. For purpose of illustration and not limitation, with reference to FIG. 3, certain outcome metrics, such as readmissions, can result in financial costs or penalties for a hospital. For example, and as embodied herein, a hospital can be assessed with a financial penalty if the rate of readmissions for the hospital are above a certain threshold or if the hospital is ranked in a lower quartile, such as the third or fourth quartile. Such penalties can be determined or estimated using a publicly-available penalty calculation, such as for penalties assessed by CMS based on certain metrics. As shown for example in FIG. 9, for purpose of illustration and not limitation, 30-day readmissions is one metric that can be improved to avoid direct penalties from CMS. Thus, identifying tactics to reduce 30-day readmissions can be beneficial to reduce such penalties. As such, as embodied herein, improving hospital performance based on metrics described herein can be beneficial to reduce financial costs and penalties assessed to the hospital.

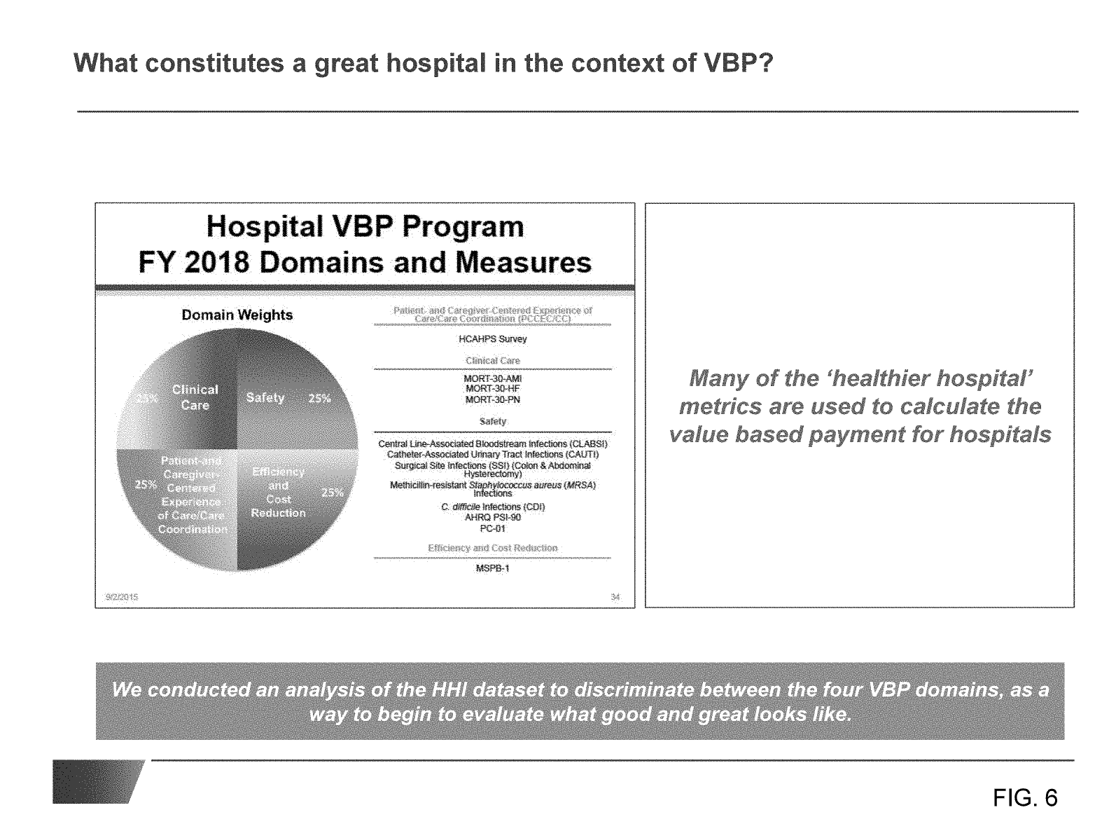

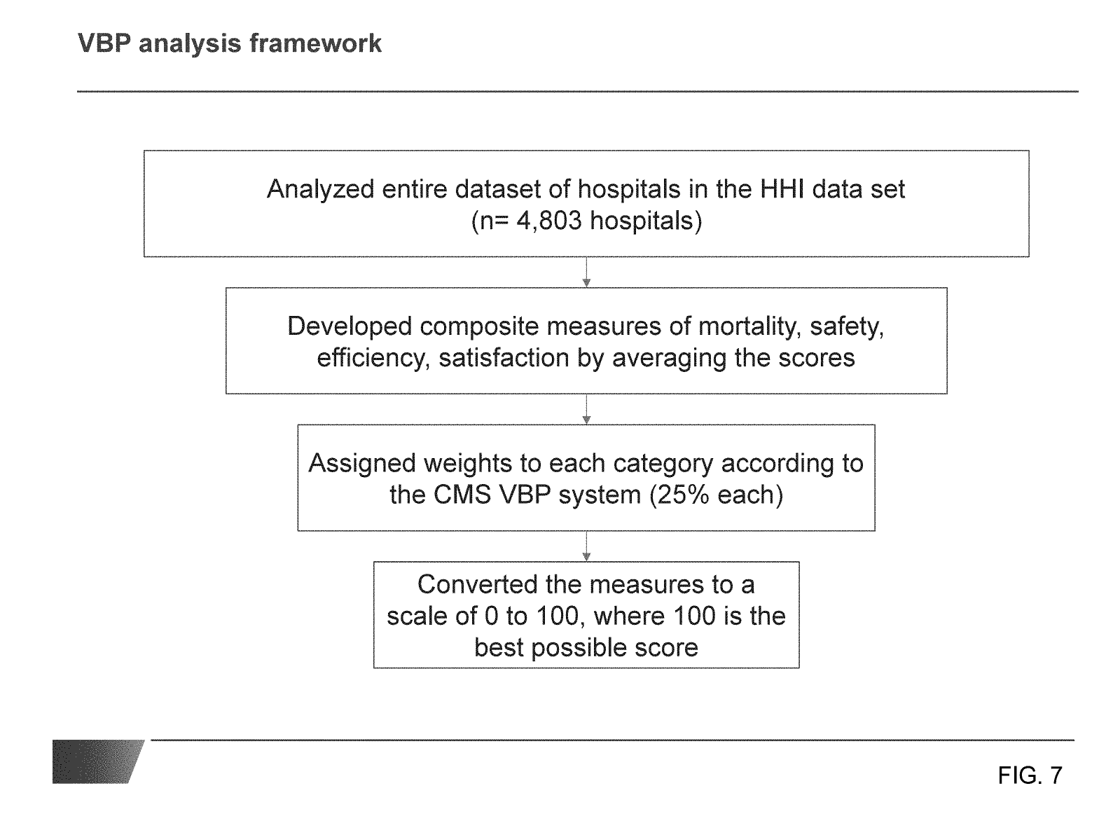

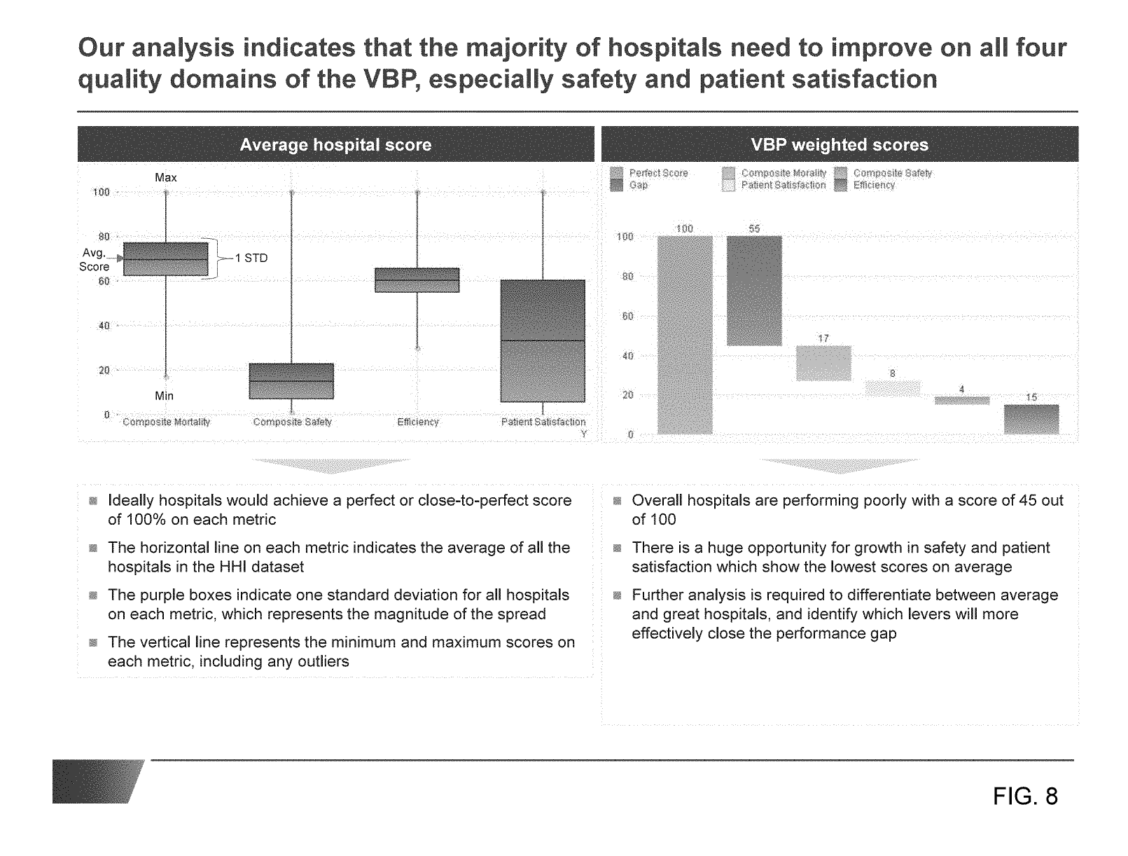

As embodied herein, hospital performance metrics described herein can be used to calculate a value-based payment (“VBP”) for hospitals, as shown for example in FIG. 6. Additional details of VBP analysis, for purpose of illustration and not limitation, are shown with reference to FIG. 7. For purpose of illustration and not limitation, and as embodied herein, VBP analysis of hospital performance metrics can include analyzing a dataset of hospital performance rankings in a database, developing composite measures, for example and without limitation, in categories of mortality, safety, efficiency and satisfaction, which can be performed by averaging scores. As embodied herein, weights can be assigned to each category, for example and as embodied herein, 25% for each category. The measures can be converted to a scale of 0 to 100, with 100 representing the best possible score. FIG. 8 illustrates an exemplary comparison of average hospital scores and corresponding VBP weighted scores determined as described herein.

Referring again to FIG. 1A, for purpose of illustration and not limitation, in a first example, at 102, method 100 includes obtaining a patient medical history information database including patient medical history information for a plurality of patients. The patient medical history information can include, for example and without limitation, and as embodied herein, for each patient, at least one medical procedure performed by a hospital of the plurality of hospitals in response to at least one of a plurality of medical indications. For purpose of illustration and not limitation, the patient medical history information includes, or be obtained from, a plurality of medical claims filed by or on behalf of the patient and/or a plurality of medical records documenting at least one medical procedure performed on the patient in response to at least one medical indication. Additionally or alternatively, and as embodied herein, the patient medical history information can be used to determine common billing codes to identify the medical procedures prescribed or performed by hospitals in the highest volumes and/or the conditions for which the highest volumes of medical claims are accrued. For purpose of illustration and not limitation, the at least one medical procedure can include a diagnostic procedure, a surgical procedure, a physical therapy, a pharmaceutical therapy, or any other medical procedure. For example, and as embodied herein, exemplary medical procedures can include using BOOST or RED discharge protocol(s) or the Intermountain Risk Score (IMRS) for patient risk stratification.

With reference to FIG. 1A, at 104, method 100 includes correlating the hospital performance ranking database with the patient medical history information database. For example, as shown in FIG. 3, hospital performance rankings can be correlated with a review of patient medical history information, such as commercial claims data, to determine a correlation of medical procedures, such as diagnostics and/or treatments, to hospital performance. For purpose of illustration and not limitation, as embodied herein, the hospital performance ranking database can be correlated with the patient medical history information database to determine, for each medical indication, a difference in a frequency of the at least one medical procedure performed by at least one hospital of the plurality of hospitals above a high ranking threshold compared to a frequency of the at least one medical procedure performed by at least one hospital of the plurality of hospitals below a low ranking threshold. Referring again to FIG. 1A, at 106, method 100 includes identifying, for each medical indication, the at least one medical procedure having the difference in the frequency above a predetermined threshold.

Referring still to FIG. 1A, in another example, which can be performed additionally in combination with, or as an alternative to, 102, 104 and 106, described above, at 103, method 100 includes obtaining a hospital interview response database including a plurality of medical procedures determined to reduce readmissions for at least one of a plurality of medical indications and, for at least one hospital of the plurality of hospitals, a response to whether the at least one hospital performs each of the plurality of medical procedures determined to reduce readmissions for the least one medical indication.

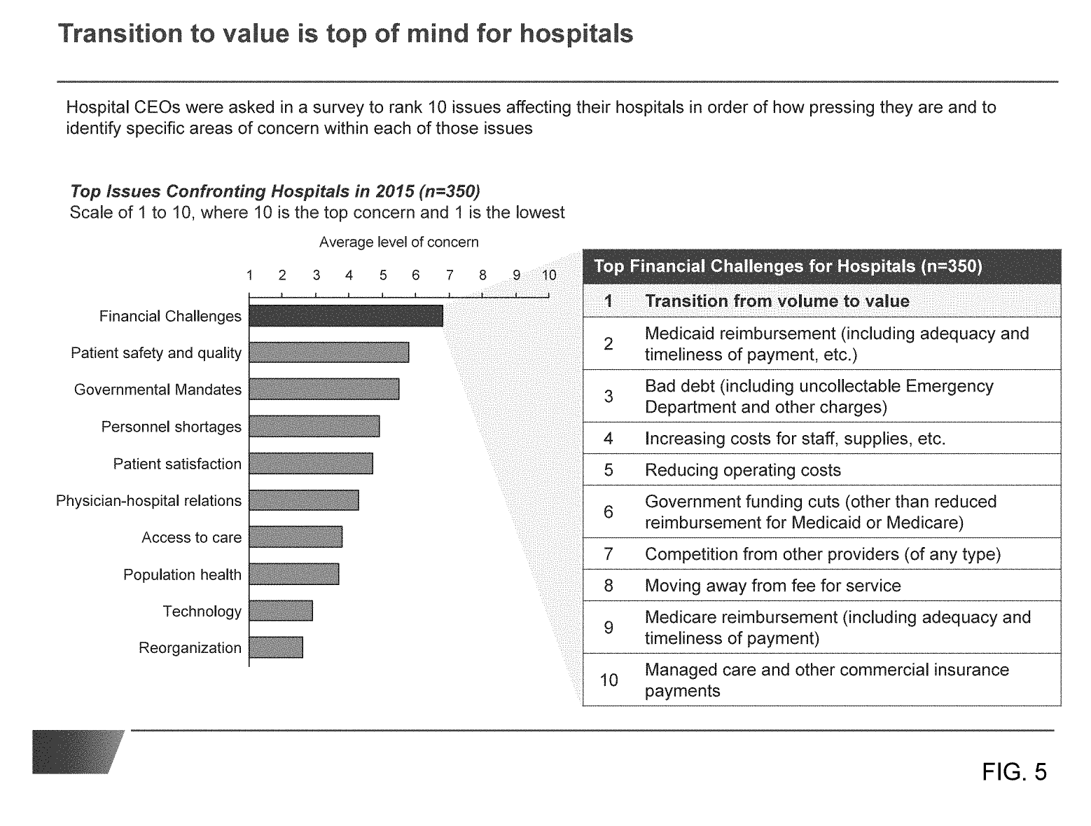

For purpose of illustration and not limitation, with reference to FIG. 3, hospital interview response data can be used to identify unmet needs and develop solutions. For example, referring now to FIG. 5, exemplary hospital response data is shown. As embodied herein, for purpose of illustration and not limitation, hospital interview response data can include a ranking of issues of concern of hospitals, and can further include a ranking of specific areas of concern within those issues.

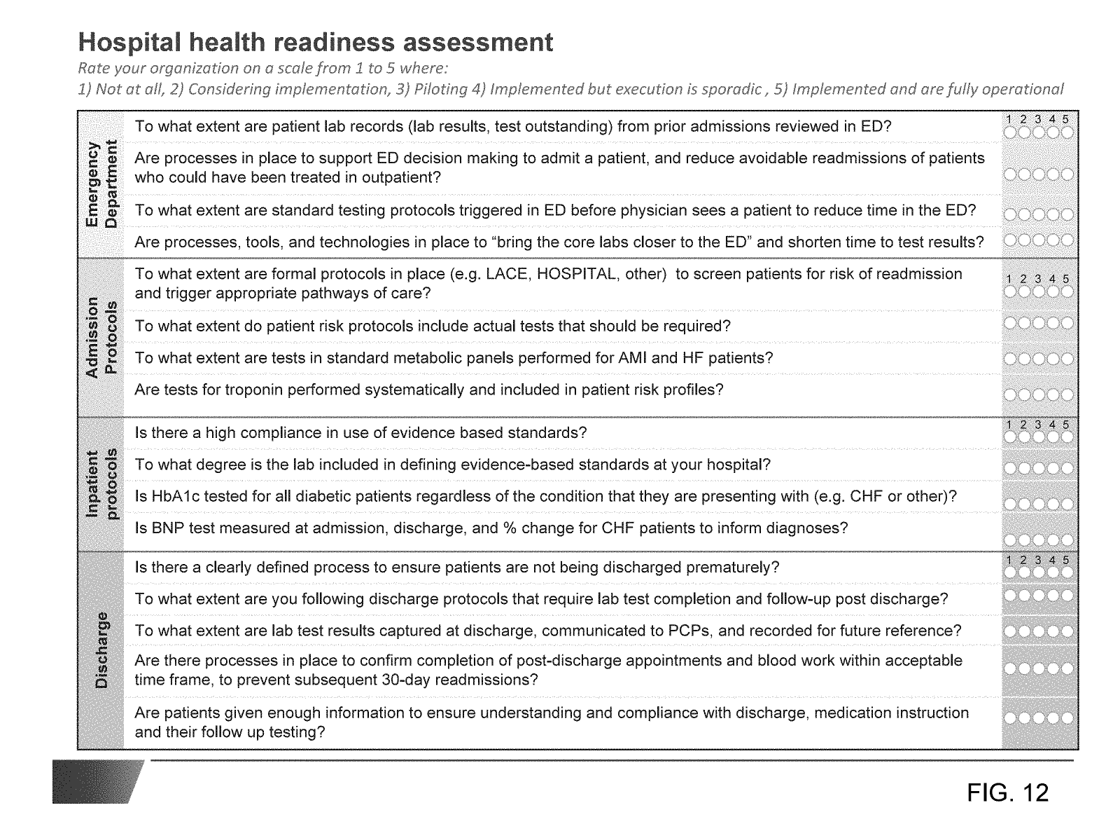

Additionally or alternatively, as shown for example in FIG. 12, hospital interview response data can include responses related to implementation of medical procedures. As embodied herein, for purpose of illustration and not limitation, the hospital interview response data can include responses related to certain categories or stages of care including, for example, emergency department protocols, admission protocols, inpatient protocols, and discharge protocols. As shown, the hospital interview response data can include a response indicating whether the hospital performs certain medical procedures determined to reduce readmissions for certain medical indications and/or in certain categories or stages of care.

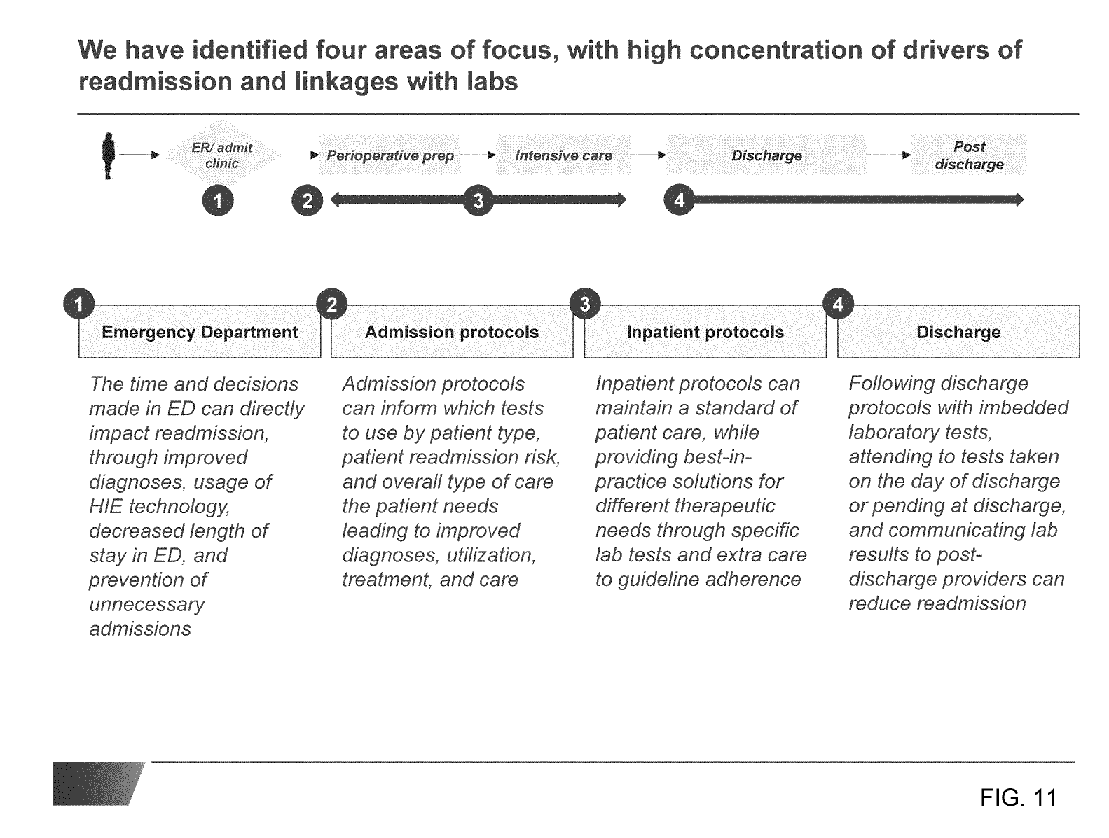

Referring again to FIG. 1A, at 105, method 100 includes identifying, from the hospital interview response database, at least one medical procedure of the plurality of medical procedures determined to reduce readmissions for the least one medical indication that is not performed by the at least one hospital. As shown for example in FIG. 11, for purpose of illustration and not limitation, medical procedures to reduce readmissions can be identified for certain medical indications and/or in certain categories or stages of care. For example, as embodied herein, the hospital interview response database can be used to identify underlying causes for a low hospital ranking in one or more metrics. For purpose of illustration and not limitation, a hospital can be identified has being in the bottom quartile of hospitals for the outcome metric of readmission rate, and a hospital interview response in the hospital interview response database can indicate that the hospital has a problem with healthcare-associated infections (HAIs) from antibiotic-resistant bacteria, such as Methicillin-resistant Staphylococcus aureus (MRSA). As such, protocols can be identified for preventing HAIs used by hospitals having low readmission rates. For example, and as embodied herein, such protocols can be identified by selecting protocols used by demographically-similar hospitals having low readmission rates. As embodied herein, demographically-similar hospitals can be determined from publicly-available data, such as U.S. census data or other suitable demographic data.

Referring now to FIG. 1B, at 108, method 100 includes selecting, for the at least one hospital, a medical indication of interest from the plurality of medical indications. Such selection can be performed, for example and without limitation, by a user via input to a graphical user interface implemented on an electronic device, such as a computer, tablet or mobile device, as described further herein. For example and not limitation, the selection can be performed by selecting a medical indication from a list of medical indications and/or by entering a medical indication into an input field.

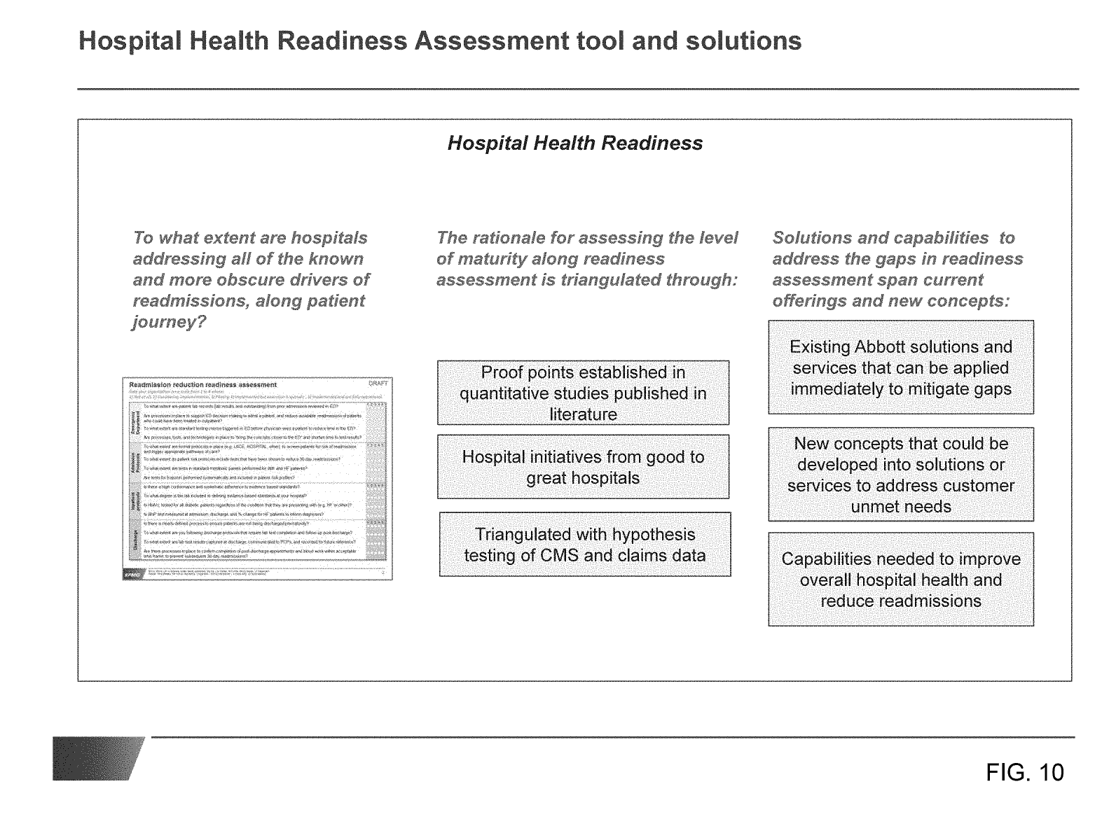

Referring still to FIG. 1B, at 110, method 100 includes presenting to a user, for the at least one hospital, at least one recommended medical procedure to improve the at least one metric related to hospital performance, the at least one recommended medical procedure selected from at least one of the at least one medical procedure having the difference in the frequency above the predetermined threshold (if 106 is performed above) and the at least one medical procedure of the plurality of medical procedures determined to reduce readmissions for the least one medical indication not performed by the at least one hospital (if 105 is performed above). FIG. 10 provides additional details related to presenting to a user at least one recommended medical procedure as described herein. Additionally or alternatively, the user can be presented, for the at least one hospital, based on the history of rankings of the hospital in the at least one metric related to hospital performance, an estimated improvement in the at least one metric related to hospital performance if the at least one recommended medical procedure is implemented, and/or, based on the sensitivity analysis, an estimated cost savings if the at least one recommended medical procedure is implemented.

Additionally or alternatively, as embodied herein, method 100 can include performing the at least one recommended medical procedure. For example and without limitation, as embodied herein, performing the at least one recommended medical procedure can include prescribing, conducting, reviewing or analyzing a diagnostic procedure; prescribing or performing a surgical procedure; prescribing or administering a pharmaceutical treatment; and/or prescribing or administering a physical therapy.

For purpose of illustration and not limitation, systems for performing the techniques described herein can include a server system, which includes applications for determining recommended medical procedures for a medical indication of interest. The server system can include one or more electronic computing devices operable to receive, transmit, process, and store data associated with the application. For example, server system can include one or more general-purpose personal computers, work stations, supercomputers, and include a processor and a memory. The hospital performance ranking database and/or the medical claim information database can be stored in the memory, and can be retrieved from, and updated into, one or more external or internal databases. Although server system is referred to as a “server,” the present disclosed subject matter contemplates the server system including any suitable type of processing device or devices. Each database, although primarily described as being a “database,” can include any suitable memory module and can take the form of volatile or non-volatile memory, including, without limitation, magnetic media, optical media, RAM, ROM, removable media, or any other suitable local or remote memory component. Any suitable number of databases can be used in conjunction with the server system.

The server system can be accessed directly by a user, or alternatively, can also include at least one user system, which can includes a display device for displaying or otherwise (via audio, audiovisual, visual, or other medium) presenting to a user the recommended medical procedure(s) for the medical indication of interest provided by the application, and a processor, a memory and network interface(s) for transmitting, formatting, manipulating information received from the server via a network or interne into a suitable format capable of being presented by the display device. The user system can also include any appropriate input devices (such as a keypad, touch screen, mouse, or other device that can accept information), output devices, mass storage media, or other suitable components for receiving, processing, storing, and communicating data. Both the input devices and output devices can include fixed or removable storage media such as a magnetic computer disk, CD-ROM, or other suitable media operable to both receive input from and provide output to a user of the user system. Additionally, the user system can include any suitable combination of software, firmware, and hardware.

In addition to the specific embodiments claimed below, the disclosed subject matter is also directed to other embodiments having any other possible combination of the dependent features claimed below and those disclosed above and in the attached figures. As such, the particular features disclosed herein can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A method for determining recommended medical procedures for a medical indication of interest, comprising:
obtaining a hospital performance ranking database including a plurality of hospitals and, for each hospital, a ranking of the hospital relative to each other hospital in at least one metric related to hospital performance;
obtaining a patient medical history information database including patient medical history information for a plurality of patients, the patient medical history information for each patient including at least one medical procedure performed by a hospital of the plurality of hospitals in response to at least one of a plurality of medical indications;
correlating the hospital performance ranking database with the patient medical history information database to determine, for each medical indication, a difference in a frequency of the at least one medical procedure performed by at least one hospital of the plurality of hospitals above a high ranking threshold compared to a frequency of the at least one medical procedure performed by at least one hospital of the plurality of hospitals below a low ranking threshold;
identifying, for each medical indication, the at least one medical procedure having the difference in the frequency above a predetermined threshold;
selecting, for the at least one hospital, a medical indication of interest from the plurality of medical indications; and
presenting to a user, for the at least one hospital, at least one recommended medical procedure to improve the at least one metric related to hospital performance, the at least one recommended medical procedure selected from the at least one medical procedure having the difference in the frequency above the predetermined threshold for the medical indication of interest.

2. The method of claim 1, wherein the hospital performance ranking database is formatted from a plurality of databases to comprise a single row of information for each hospital of the plurality of hospitals.

3. The method of claim 1, wherein the at least one metric includes at least one outcome metric, patient satisfaction metric, clinical care metric, patient safety metric, and hospital operations metric.

4. The method of claim 3, wherein the at least one outcome metric includes at least one of morality, readmissions, and complications.

5. The method of claim 1, wherein the ranking of each hospital corresponds to one of four quartiles.

6. The method of claim 1, further comprising providing a database of best practice information, including, for each of the hospitals of the plurality of hospitals, whether the hospital performs one or more best practices, wherein the hospital is further ranked based at least in part on whether the hospital performs one or more best practices.

7. The method of claim 6, further comprising presenting to a user recommendations for improving identified areas of need based on the database of best practices.

8. The method of claim 1, further comprising identifying areas of need based on sensitivity analysis of metrics for which the hospital is performing below average.

9. The method of claim 1, further comprising identifying areas of need based on questions related to one or more hospital best practices.

10. A method for determining recommended medical procedures for a medical indication of interest, comprising:
obtaining a hospital performance ranking database including a plurality of hospitals and, for each hospital, a ranking of the hospital relative to each other hospital in at least one metric related to hospital performance;
obtaining a hospital interview response database including a plurality of medical procedures determined to reduce readmissions for at least one of a plurality of medical indications and, for at least one hospital of the plurality of hospitals, a response to whether the at least one hospital performs each of the plurality of medical procedures determined to reduce readmissions for the least one medical indication;
identifying, from the hospital interview response database, at least one medical procedure of the plurality of medical procedures determined to reduce readmissions for the least one medical indication that is not performed by the at least one hospital;
selecting, for the at least one hospital, a medical indication of interest from the plurality of medical indications; and
presenting to a user, for the at least one hospital, at least one recommended medical procedure to improve the at least one metric related to hospital performance, the at least one recommended medical procedure selected from the at least one medical procedure of the plurality of medical procedures determined to reduce readmissions for the least one medical indication that is not performed by the at least one hospital.

11. A method for determining recommended medical procedures for a medical indication of interest, comprising:
obtaining a hospital performance ranking database including a plurality of hospitals and, for each hospital, a ranking of the hospital relative to each other hospital in at least one metric related to hospital performance;
obtaining a patient medical history information database including patient medical history information for a plurality of patients, the patient medical history information for each patient including at least one medical procedure performed by a hospital of the plurality of hospitals in response to at least one of a plurality of medical indications;
obtaining a hospital interview response database including a plurality of medical procedures determined to reduce readmissions for at least one of a plurality of medical indications and, for at least one hospital of the plurality of hospitals, a response to whether the at least one hospital performs each of the plurality of medical procedures determined to reduce readmissions for the least one medical indication;
correlating the hospital performance ranking database with the patient medical history information database to determine, for each medical indication, a difference in a frequency of the at least one medical procedure performed by at least one hospital of the plurality of hospitals above a high ranking threshold compared to a frequency of the at least one medical procedure performed by at least one hospital of the plurality of hospitals below a low ranking threshold;
identifying, for each medical indication, the at least one medical procedure having the difference in the frequency above a predetermined threshold;
identifying, from the hospital interview response database, at least one medical procedure of the plurality of medical procedures determined to reduce readmissions for the least one medical indication that is not performed by the at least one hospital;
selecting, for the at least one hospital, a medical indication of interest from the plurality of medical indications; and
presenting to a user, for the at least one hospital, at least one recommended medical procedure to improve the at least one metric related to hospital performance, the at least one recommended medical procedure selected from at least one of the at least one medical procedure having the difference in the frequency above the predetermined threshold for the medical indication of interest and the at least one recommended medical procedure selected from the at least one medical procedure of the plurality of medical procedures determined to reduce readmissions for the least one medical indication that is not performed by the at least one hospital.
